(19) United States
(12) Patent Application Publication
(10) Pub. No.: US 2025/0161284 A1
(43) Pub. Date: May 22, 2025

(54) USES OF BUPIVACAINE MULTIVESICULAR LIPOSOMES AS STELLATE GANGLION BLOCK FOR TREATING DISORDERS ASSOCIATED WITH SYMPATHETIC NERVOUS SYSTEM

(71) Applicant: Pacira Pharmaceuticals, Inc., San Diego, CA (US)

(72) Inventors: John Spranger, Hingham, MA (US); Roy Winston, Tampa, FL (US); Jonathan Slonin, Palm City, FL (US)

(21) Appl. No.: 18/949,639

(22) Filed: Nov. 15, 2024

Related U.S. Application Data
(60) Provisional application No. 63/599,786, filed on Nov. 16, 2023.

(51) Int. Cl. A61K 31/445; A61K 9/00; A61K 9/127

(52) U.S. Cl. CPC A61K 31/445; A61K 9/0019; A61K 9/127

(57) ABSTRACT

Embodiments of the present disclosure relate to methods of using bupivacaine multivesicular liposomes (MVLs) as stellate ganglion block (SGB) for treating or ameliorating a sympathetic nervous system disorder associated with overactive or unbalanced sympathetic nervous system, including but not limited to, Raynaud's syndrome, post-mastectomy pain, reflex sympathetic dystrophy (RSD), phantom limb pain, hyperhidrosis, trigeminal neuralgia/facial pain, scleroderma, and long COVID.

### INCORPORATION BY REFERENCE TO ANY PRIORITY APPLICATIONS

The present application claims the benefit of priority to U.S. Provisional Application No. 63/599,786, filed Nov. 16, 2023, which is incorporated by reference in its entirety.

### BACKGROUND

### Field

The present disclosure relates to compositions of bupivacaine multivesicular liposomes (MVLs) and uses thereof as stellate ganglion block for treating disorders associated with sympathetic nervous system.

### Description of the Related Art

The stellate ganglion is formed by a fusion of the C7, C8, and T1 regions (for example, the C7, C8, and T1 ganglia) of the paravertebral chain. Bupivacaine is a versatile drug that has been shown to be efficacious for a wide variety of indications, including: postoperative analgesia, peripheral nerve blocking, diagnostic and therapeutic procedures, and obstetrical procedures. See Obaid Malik, et al., Emerging roles of liposomal bupivacaine in anesthesia practice, 33 Journal of Anaesthesiology Clinical Pharmacology, 151 (2017).

### SUMMARY

Some embodiments of the present disclosure relate to a method of treating a patient who has suffered from or is at risk of suffering from a sympathetic nervous system disorder, including: administering a composition comprising an effective amount of bupivacaine multivesicular liposomes (MVLs) to one or more nerves of the stellate ganglion of the patient, or an autonomic tissue area peripheral to the stellate ganglion. In some embodiments, the method further comprises identifying or selecting a patient who has suffered from or is at risk of suffering from a disorder associated with sympathetic nervous system.

Some embodiments of the present disclosure relate to a method of treating or ameliorating an overactive or unbalanced sympathetic nervous system associated disorder in a patient in need thereof, including: administering a composition comprising an effective amount of bupivacaine MVLs to one or more nerves of the stellate ganglion of the patient, or an autonomic tissue area peripheral to the stellate ganglion. In some embodiments, the method further comprises identifying or selecting a patient who has suffered from or is at risk of suffering from the overactive or unbalanced sympathetic nervous system.

Some embodiments of the present disclosure relate to a method of reducing or interrupting sympathetic nervous system activity associated with a sympathetic nervous system disorder in a patient in need thereof, including: administering a composition comprising an effective amount of bupivacaine MVLs to one or more nerves of the stellate ganglion of the patient, or an autonomic tissue area peripheral to the stellate ganglion. In some embodiments, the method further comprises identifying or selecting a patient who has suffered from or is at risk of suffering from overactive or overstimulated sympathetic nervous system activity.

In any embodiments of the methods described herein, the sympathetic nervous system disorder can be Raynaud's syndrome, post-mastectomy pain, reflex sympathetic dystrophy (RSD), phantom limb pain, hyperhidrosis, trigeminal neuralgia/facial pain, scleroderma, or long COVID. In some embodiments, the sympathetic nervous system disorder is Raynaud's syndrome. In some embodiments, the sympathetic nervous system disorder is post-mastectomy pain. In some embodiments, the sympathetic nervous system disorder is reflex sympathetic dystrophy (RSD). In some embodiments, the sympathetic nervous system disorder is phantom limb pain. In some embodiments, the sympathetic nervous system disorder is hyperhidrosis. In some embodiments, the sympathetic nervous system disorder is trigeminal neuralgia/facial pain. In some embodiments, the sympathetic nervous system disorder is scleroderma. In some embodiments, the sympathetic nervous system disorder is long COVID. In some embodiments, the method results in temporary interruption of sympathetic nervous system activity associated with the sympathetic nervous system disorder. In some embodiments, the method causes a temporary reduction in the ability of the sympathetic nervous system to release neurotransmitters. In some embodiments, the one or more nerves of the stellate ganglion comprise one or more nerves that provide sympathetic nervous system activity associated with sympathetic nervous system disorders.

In some embodiments of the methods described herein, the concentration of bupivacaine in the composition is from about 1 mg/mL to about 30 mg/mL, or from about 2 mg/mL to about 20 mg/mL. In one embodiment, the concentration of bupivacaine in the composition is about 13.3 mg/mL. In some embodiments, the effective amount of bupivacaine administered is from about 10 mg to about 200 mg, from about 20 mg to about 100 mg, or from about 25 mg to about 75 mg.

In some embodiments of the methods described herein, the total volume of the composition administered is from about 1 mL to about 20 mL. In some embodiments, the total volume of the composition administered is from about 5 mL to about 10 mL for unilateral stellate ganglion block. In further embodiments, the total volume of the composition administered is from about 10 mL to about 20 mL for bilateral stellate ganglion block. In some embodiments, bupivacaine is in a salt form. In further embodiments, the bupivacaine is in the form of bupivacaine phosphate.

In some embodiments of the methods described herein, the administering temporarily reduces or interrupts sympathetic stimulation to the nervous system. In some embodiments, the temporary reduction or interruption of sympathetic stimulation to the nervous system is for up to 3 days, 7 days, 10 days, 14 days, 21 days, or 1, 2, 3, 4, 5 or 6 months.

In any embodiment of the methods described herein, the administering of the composition of bupivacaine MVLs comprises:
navigating a treatment apparatus to stellate ganglion of the patient, or an autonomic tissue area peripheral to the stellate ganglion, wherein the treatment apparatus comprises a needle and a compartment for the composition of bupivacaine MVLs;
injecting bupivacaine MVLs from the treatment apparatus to one or more nerves of the stellate ganglion, or the autonomic tissue area peripheral to the stellate ganglion; and
removing the treatment apparatus from the patient.

In some embodiments, navigating the treatment apparatus to the stellate ganglion comprises inserting the needle of the treatment apparatus percutaneously to one or more nerves of the stellate ganglion area. In some embodiments, navigating the treatment apparatus to the stellate ganglion comprises inserting the needle of the treatment apparatus percutaneously to one or more nerves of the stellate ganglion, or one or more nerves of the autonomic tissue area peripheral to the stellate ganglion, or both. In further embodiments, navigating the treatment apparatus to the stellate ganglion comprises guiding the needle using an imaging guide. In further embodiments, the imaging guide comprises fluoroscopic or ultrasound imaging guide.

In some embodiments of the methods described herein, the injection comprises bolus injection, continuous infusion, or a combination thereof. In some embodiments, the administering is to the left stellate ganglion. In some embodiments, the administering is to both the left and the right stellate ganglion. In some embodiments, the one or more desired nerves of the stellate ganglion or the autonomic tissue area peripheral to the stellate ganglion comprise one or more desired nerves of the autonomic tissue area peripheral to the stellate ganglion. In some other embodiments, the one or more desired nerves of the stellate ganglion or the autonomic tissue area peripheral to the stellate ganglion comprise one or more desired nerves of the autonomic tissue area peripheral to the stellate ganglion. In some other embodiments, the one or more desired nerves of the stellate ganglion or the autonomic tissue area peripheral to the stellate ganglion comprise one or more desired nerves of a paravertebral chain. In some such embodiments, the one or more desired nerves of the paravertebral chain comprises one or more desired nerves of one or more of a C7 region of the paravertebral chain, a C8 region of the paravertebral chain, a T1 region of the paravertebral chain, or a T2 region of the paravertebral chain, or combinations thereof. In some further embodiments, the one or more desired nerves of the paravertebral chain comprise one or more desired nerves of the T1 region of a paravertebral chain or the T2 region of the paravertebral chain, or a combination thereof.

### DETAILED DESCRIPTION

Embodiments of the present disclosure relate to methods of using bupivacaine multivesicular liposomes (MVLs) as stellate ganglion block (SGB) for treating or managing conditions associated with the sympathetic nervous system, such as sympathetic nervous system disorders, such as Raynaud's syndrome, post-mastectomy pain, reflex sympathetic dystrophy (RSD), phantom limb pain, hyperhidrosis, trigeminal neuralgia/facial pain, scleroderma, or long COVID.

The section headings used herein are for organizational purposes only and are not to be construed as limiting the subject matter described.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, formulation, or device, the term “comprising” means that the compound, composition, formulation, or device includes at least the recited features or components, but may also include additional features or components.

As used herein, the terms “bupivacaine encapsulated multivesicular liposomes”, or “bupivacaine MVLs” refer to a multivesicular liposome composition encapsulating bupivacaine. In some embodiments, the composition is a pharmaceutical formulation, where the bupivacaine encapsulated multivesicular liposome particles are suspended in a liquid suspending medium to form a suspension. In some such embodiments, the bupivacaine-MVL suspension may also include free or unencapsulated bupivacaine. In some cases, the free or unencapsulated bupivacaine may be less than about 8%, 7%, 6%, 5%, 4%, 3%, 2%, 1%, 0.5%, 0.2% or 0.1%, by weight of the total amount of the bupivacaine in the composition, or in a range defined by any of the two preceding values. In some embodiments, the free bupivacaine may be about 5% or less by weight of the total amount of the bupivacaine in the composition. In further embodiments, the free bupivacaine may be about 8% or less during the shelf life of the product (i.e., up to 2 years when stored at 2-8° C.).

As used herein, the term “encapsulated” means that bupivacaine is inside a liposomal particle, for example, the MVL particles. In some instances, bupivacaine may also be on an inner surface, or intercalated in a membrane, of the MVLs.

As used herein, the term “unencapsulated bupivacaine” or “free bupivacaine” refers to bupivacaine outside the liposomal particles, for example the MVL particles. For example, unencapsulated bupivacaine may reside in the suspending solution of these particles.

As used herein, “effective amount” or “pharmaceutically effective amount” refers to an amount of therapeutic agent that has a therapeutic effect. The dosages of a pharmaceutically active ingredient which are useful in treatment are therapeutically effective amounts. Thus, as used herein, a therapeutically effective amount means an amount of therapeutic agent that produces the desired therapeutic effect as judged by clinical trial results and/or model animal studies.

“Treat,” “treatment,” or “treating,” as used herein refers to administering a pharmaceutical composition/formulation for prophylactic and/or therapeutic purposes. The term “prophylactic treatment” refers to treating a patient who is not yet suffering from a disease, but who is susceptible to, or otherwise at risk of, a particular disease, whereby the treatment reduces the likelihood that the patient will develop the disease. The term “therapeutic treatment” refers to administering treatment to a patient already suffering from a disease.

### Bupivacaine Multivesicular Liposomes

Some aspect of the present disclosure relates to a composition of bupivacaine encapsulated multivesicular liposomes (MVLs), comprising: bupivacaine residing inside a plurality of internal aqueous chambers of the MVLs separated by lipid membranes, wherein the lipid membranes comprise 1,2-dierucoylphosphatidylcholine (DEPC), 1,2-dipalmitoyl-sn-glycero-3-phospho-rac-(1-glycerol) (DPPG), and at least one neutral lipid; and an aqueous medium in which the bupivacaine MVLs are suspended.

In some embodiments, the composition of bupivacaine MVLs may have a volume of 10 mL or 20 mL for a single dose administration. In some embodiments, the percent packed particle volume (% PPV) of the composition of bupivacaine MVLs is about 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64% or 65%. In some such embodiments, the concentration of the bupivacaine in the composition is from about 11 mg/mL to about 17 mg/mL. In one embodiment, the concentration of the bupivacaine in the composition is about 13.3 mg/mL. In other embodiments, the concentration of the bupivacaine in the composition is from about 18 mg/mL to about 22 mg/mL, or about 20 mg/mL. In further embodiments, the composition comprises less than 5%, 4%, 3%, 2% or 1% unencapsulated bupivacaine, wherein the amount of unencapsulated bupivacaine is calculated based on the total weight of the bupivacaine in the composition. In some embodiments, the d₅₀ of the multivesicular liposomes in the composition is about 24 μm to about 31 μm. In one embodiment, the d₅₀ of the multivesicular liposomes in the composition is about 27 μm. In any embodiments, the bupivacaine MVLs used can be Exparel®, which is 13.3 mg bupivacaine MVL liposomes approved by the FDA and marketed by Pacira Pharmaceuticals, Inc.

### Pharmaceutical Compositions

In some embodiments, the composition comprising bupivacaine MVLs is a pharmaceutical formulation including a pharmaceutically acceptable carrier. Effective injectable bupivacaine MVL compositions can be in a liquid suspension form. Such injectable suspension compositions include a liquid suspending medium, with or without adjuvants, as a vehicle. The suspending medium can be, for example, aqueous solutions of sodium chloride (i.e., saline solution), dextrose, sucrose, polyvinylpyrrolidone, polyethylene glycol, a pH modifying agent described herein, or combinations of the above. In some embodiments, the suspending medium of bupivacaine MVLs is a saline solution, optionally containing a tonicity agent such as dextrose and/or a pH modifying agent such as lysine. In other embodiments, the suspending medium of bupivacaine MVLs is a buffered saline solution (e.g., containing a phosphate buffer).

Suitable physiologically acceptable storage solution components are used to keep the compound suspended in suspension compositions. The storage solution components can be chosen from thickeners such as carboxymethylcellulose, polyvinylpyrrolidone, gelatin and alginates. Many surfactants are also useful as suspending agents. The suspending medium can also contain lecithin, alkylphenol polyethylene oxide adducts, naphthalenesulfonates, alkylbenzenesulfonates, or polyoxyethylene sorbitan esters. In some embodiments, the bupivacaine MVL composition is free or substantially free of any additive of preservatives.

In any embodiments of the composition of bupivacaine MVLs described herein, the composition may be a pharmaceutical composition suitable for human administration. In further embodiments, the composition may be an aqueous suspension of bupivacaine MVL particles. In some embodiments, the administered bupivacaine is in a salt form. In further embodiments, the administered bupivacaine is in the form of bupivacaine phosphate.

### Methods of Treatment

Some embodiments of the present disclosure relate to a method of treating a patient who has suffered from or is at risk of suffering from a sympathetic nervous system disorder, comprising administering a composition containing bupivacaine multivesicular liposomes (MVLs) as described herein. Some embodiments of the present disclosure relate to a method of treating or ameliorating an overactive or unbalanced sympathetic nervous system associated disorder in a patient in need thereof, comprising administering a composition containing bupivacaine MVLs. The present embodiments also provide a method of reducing or interrupting sympathetic stimulation to the nervous system of a patient in need thereof, comprising administering a composition containing bupivacaine MVLs. In some embodiments, the methods disclosed herein include administering a composition containing bupivacaine MVLs to one or more nerves of stellate ganglion of the patient, or one or more nerves of an autonomic tissue area peripheral to the stellate ganglion, or a combination thereof. In some embodiments, the methods include identifying or selecting a patient who has suffered from or is at risk of suffering from a sympathetic nervous system disorder. In some embodiments, the methods include identifying or selecting a patient who has suffered from or is at risk of suffering from the overactive or unbalanced sympathetic nervous system. In some embodiments, the methods include identifying or selecting a patient who has suffered from or is at risk of suffering from overactive or overstimulated sympathetic nervous system. In some embodiments of the methods disclosed herein, the administration of the bupivacaine MVLs to one or more nerves of stellate ganglion of the patient, or autonomic tissue area peripheral to the stellate ganglion, temporarily reduces or interrupts sympathetic stimulation to the nervous system for a sustained period of time. In some embodiments, the temporary reduction or interruption of sympathetic stimulation to the nervous system is for up to 3 days, 7 days, 10 days, 14 days, 21 days, 1 month, 2 months, 3 months, 4 months, 5 months or 6 months.

Certain embodiments employ methods to at least temporarily interrupt the activity of certain nerves of the stellate ganglion and/or autonomic tissue peripheral to the stellate ganglion. Certain embodiments employ methods to the stellate ganglion and/or autonomic tissue peripheral to the stellate ganglion to perform a stellate ganglion block. Methods of the present disclosure may, for example, prevent the release of one or more neurotransmitters that may cause an undesirable effect. In some embodiments, the methods disclosed herein may prevent the release of neurotransmitters (for example, catecholamines, such as epinephrine and norepinephrine) that may cause sympathetic nervous system disorders. In some embodiments, the methods disclosed herein may allow for treatment of sympathetic nervous system disorders for which psychotropics are not effective. In some embodiments, methods of treatment of the nerves of the stellate ganglion and/or autonomic tissue peripheral to the stellate ganglion can be used to treat sympathetic nervous system disorders such as Raynaud's syndrome, post-mastectomy pain, reflex sympathetic dystrophy (RSD), phantom limb pain, hyperhidrosis, trigeminal neuralgia/facial pain, scleroderma, and long COVID.

In certain embodiments, the methods described herein can be used to block or reduce nervous system activity associated with sympathetic nervous system disorders.

In certain embodiments, the methods described herein include administering a composition comprising an effective amount of bupivacaine multivesicular liposomes (MVLs) to one or more nerves of stellate ganglion of the patient, or an autonomic tissue area peripheral to the stellate ganglion.

In certain embodiments, sympathetic nervous system activity associated with sympathetic nervous system disorders can be affected by the paravertebral chain such as for example, the C7, C8, T1, and T2 regions (for example, the C7, C8, T1, and T2 paravertebral ganglia) of the paravertebral chain. In some patients, the C7, C8, T1, and T2 paravertebral ganglia are fused to form the stellate ganglion. Sympathetic nervous system activity associated with sympathetic nervous system disorders may also be affected by several other regions of the sympathetic nervous system peripheral to the stellate ganglion. For example, in some patients, the T2 paravertebral ganglia is not part of the stellate ganglion. In some embodiments, the methods described herein can be employed on the one or more autonomic tissue regions peripheral to the stellate ganglion, such as the T2 paravertebral ganglia in patients in which it does not form part of the stellate ganglion to block or reduce sympathetic nervous system activity associated with sympathetic nervous system disorders. In some embodiments, the methods described herein can be employed on one or more of the T1 region of the paravertebral chain, the T2 region of the paravertebral chain, and the stellate ganglion in order to block or reduce sympathetic stimulation to one or more brain regions associated with sympathetic nervous system disorders. In some embodiments, the systems and methods described herein can be used to perform cryogenic therapy on one or more of the T1 region of the paravertebral chain, the T2 region of the paravertebral chain, and the stellate ganglion in order to block or reduce sympathetic nervous system activity associated with sympathetic nervous system disorders.

In some embodiments, the methods described herein can be used to reduce sympathetic nervous system activity associated with sympathetic nervous system disorders or the ability of the sympathetic nervous system to perform sympathetic nervous system activity associated with sympathetic nervous system disorders by at least 10%, 15%, 20%, 25%, 30%, 35%, 40%, 45%, 50%, 55%, 60%, 65%, 70%, 75%, 80%, 85%, or 90%, or any other suitable amount or degree, when compared to the administration of the same dose amount of bupivacaine in an immediate release composition. In some embodiments, the systems and methods described herein can be used to reduce sympathetic nervous system activity associated with sympathetic nervous system disorders or the ability of the sympathetic nervous system to perform sympathetic nervous system activity associated with sympathetic nervous system disorders by between 25% and 75%, between 35% and 65%, between 25% and 50%, between 50% and 75%, or any other suitable ranges.

In some embodiments, the methods described herein can be used to reduce innervation to one or more anatomical regions associated with sympathetic nervous system disorders by at least 10%, 15%, 20%, 25%, 30%, 35%, 40%, 45%, 50%, 55%, 60%, 65%, 70%, 75%, 80%, 85%, or 90%, or any other suitable amount or degree, when compared to the administration of the same dose amount of bupivacaine in an immediate release composition. In some embodiments, the methods described herein can be used to reduce innervation to one or more anatomical regions associated with sympathetic nervous system disorders by between 25% and 75%, between 35% and 65%, between 25% and 50%, between 50% and 75%, or any other suitable range.

In some embodiments, the methods described herein can be used to reduce the release of catecholamines associated with sympathetic nervous system disorders or the ability of the sympathetic nervous system to release catecholamines associated with sympathetic nervous system disorders by at least 10%, 15%, 20%, 25%, 30%, 35%, 40%, 45%, 50%, 55%, 60%, 65%, 70%, 75%, 80%, 85%, or 90%, or any other suitable amount, when compared to the administration of the same dose amount of bupivacaine in an immediate release composition. In some embodiments, the methods described herein can be used to reduce the release of catecholamines associated with sympathetic nervous system disorders or the ability of the sympathetic nervous system to release catecholamines associated with sympathetic nervous system disorders by between 25% and 75%, between 35% and 65%, between 25% and 50%, between 50% and 75%, or any other suitable range. The catecholamines may include norepinephrine and/or epinephrine.

In some embodiments, the methods described herein can be used to cause axonotmesis of at least 10%, 15%, 20%, 25%, 30%, 35%, 40%, 45%, 50%, 55%, 60%, 65%, 70%, 75%, 80%, 85%, or 90% of the nerves that provide sympathetic nervous system activity associated with one or more sympathetic nervous system disorders (e.g., sympathetic stimulation to one or more anatomical regions associated with sympathetic nervous system disorders), or any other suitable amount or degree, as compared to the amount or degree of axonotmesis of the nerves that provide sympathetic nervous system activity associated with one or more sympathetic nervous system disorders (e.g., sympathetic stimulation to the one or more anatomical regions associated with sympathetic nervous system disorders) when the administration of the same dose amount of bupivacaine is in an immediate release composition. In some embodiments, the methods described herein can be used to cause axonotmesis of between 25% and 75%, between 35% and 65%, between 25% and 50%, or between 50% and 75% of the nerves that provide sympathetic nervous system activity associated with one or more sympathetic nervous system disorders (e.g., sympathetic stimulation to one or more anatomical regions associated with sympathetic nervous system disorders), or any other suitable range.

In some embodiments, the methods described herein can be used to cause axonotmesis of at least 10%, 15%, 20%, 25%, 30%, 35%, 40%, 45%, 50%, 55%, 60%, 65%, 70%, 75%, 80%, 85%, or 90% of the nerves of the stellate ganglion and/or autonomic tissue peripheral to the stellate ganglion, or any other suitable amount or degree, as compared to the amount or degree of axonotmesis of the nerves of the stellate ganglion and/or autonomic tissue peripheral to the stellate ganglion when the administration of the same dose amount of bupivacaine is in an immediate release composition. In some embodiments, the methods described herein can be used to cause axonotmesis of between 25% and 75%, between 35% and 65%, between 25% and 50%, or between 50% and 75% of the nerves of the stellate ganglion, or any other suitable range.

Disabling the nerves of the stellate ganglion and/or autonomic tissue peripheral to the stellate ganglion using the methods disclosed herein can provide for prophylactic treatment to prevent various disorders, such as sympathetic nervous system disorders, by temporarily preventing the release of certain neurotransmitters. Disabling the nerves of the stellate ganglion and/or autonomic tissue peripheral to the stellate ganglion using the methods disclosed herein may act as an alternative to a local anesthetic stellate ganglion block.

In certain embodiments, disabling the nerves of the stellate ganglion and/or autonomic tissue peripheral to the stellate ganglion using the methods disclosed herein can be used to treat a patient who has suffered from or is at risk of suffering from sympathetic nervous system disorders such as Raynaud's syndrome, post-mastectomy pain, reflex sympathetic dystrophy (RSD), phantom limb pain, hyperhidrosis, trigeminal neuralgia/facial pain, scleroderma, and long COVID.

The methods described herein can allow for both short-term and long-term therapy for sympathetic nervous system disorders. In certain embodiments, the axonotmesis of the nerve(s) using the methods described herein can provide for relief for sympathetic nervous system disorders for an extended period of time (for example, up to 2 weeks, 3 weeks, 1 month, 2 months, 3 months, 4 months, 5 months, or 6 months). In certain embodiments, the methods described herein can advantageously be used to remodel the nerve(s) of the stellate ganglion and/or autonomic tissue peripheral to the stellate ganglion. For example, certain embodiments can cause axonotmesis of the nerve(s) of the stellate ganglion and/or autonomic tissue peripheral to the stellate ganglion, and the axon and myelin sheath can regenerate post-treatment. In certain embodiments, the nerve(s) may regenerate with a desired change in structure and/or function. For example, in some embodiments, the nerves can regenerate with a modified structure and/or function such that the treated condition no longer occurs following regeneration. For example, in certain embodiments, following regeneration, innervation may be reduced so that the previous condition no longer occurs. In certain embodiments, the nerves may be regenerated with new sodium channels having improved anatomical structures and/or function.

### Patients

In some embodiments, the present disclosure includes identifying or selecting a patient to benefit from the treatments with bupivacaine MVLs as described herein. In some embodiments, the patient suffers from a disorder associated with sympathetic nervous system. In further embodiments, the patient has suffered from or is at risk of suffering from a disorder associated with sympathetic nervous system. In some embodiments, the patient is in need of treating or ameliorating an overactive or unbalanced sympathetic nervous system. In further embodiments, the patient has suffered from or is at risk of suffering from an overactive sympathetic nervous system. In some embodiments, the patient is in need of reducing or interrupting sympathetic nervous system activity associated with a sympathetic nervous system disorder.

In some embodiments, the patient is in need of reducing or interrupting sympathetic nervous system activity to the nervous system. In further embodiments, the patient has suffered from or is at risk of suffering from overactive or overstimulated sympathetic nervous system that are associated with one or more conditions of a sympathetic nervous system disorder. In some embodiments, the sympathetic nervous system disorder is sympathetic nervous system disorders such as Raynaud's syndrome, post-mastectomy pain, reflex sympathetic dystrophy (RSD), phantom limb pain, hyperhidrosis, trigeminal neuralgia/facial pain, scleroderma, long COVID, or combinations thereof. In some embodiments, the sympathetic nervous system disorder is Raynaud's syndrome. In some embodiments, the sympathetic nervous system disorder is post-mastectomy pain. In some embodiments, the sympathetic nervous system disorder is reflex sympathetic dystrophy (RSD). In some embodiments, the sympathetic nervous system disorder is phantom limb pain. In some embodiments, the sympathetic nervous system disorder is hyperhidrosis. In some embodiments, the sympathetic nervous system disorder is trigeminal neuralgia/facial pain. In some embodiments, the sympathetic nervous system disorder is scleroderma. In some embodiments, the sympathetic nervous system disorder is long COVID.

In some embodiments, the patient has suffered from or is at risk of suffering from Raynaud's syndrome. In some embodiments, the patient has suffered from or is at risk of suffering from post-mastectomy pain. In some embodiments, the patient has suffered from or is at risk of suffering from reflex sympathetic dystrophy (RSD). In some embodiments, the patient has suffered from or is at risk of suffering from phantom limb pain. In some embodiments, the patient has suffered from or is at risk of suffering from hyperhidrosis. In some embodiments, the patient has suffered from or is at risk of suffering from trigeminal neuralgia/facial pain. In some embodiments, the patient has suffered from or is at risk of suffering from scleroderma. In some embodiments, the patient has suffered from or is at risk of suffering from long COVID.

### Dosage

In some embodiments of the methods disclosed herein, the concentration of bupivacaine in the composition is about 1 mg/mL, 5 mg/mL, 10 mg/mL, 15 mg/mL, 20 mg/mL, 25 mg/mL, or 30 mg/mL, or a range defined by any two of the preceding values. In some embodiments, the concentration of bupivacaine in the composition is from about 1 mg/mL to about 30 mg/mL. In some embodiments, the concentration of bupivacaine in the composition is from about 1 mg/mL to about 30 mg/mL, from about 2 mg/mL to about 25 mg/mL, from about 5 mg/mL to about 20 mg/mL, or from about 10 mg/mL to about 15 mg/mL. In some embodiments, the concentration of bupivacaine in the composition is from about 12 mg/mL to about 14 mg/mL. In some embodiments, the concentration of bupivacaine in the composition is about 13.3 mg/mL. In other embodiments, the concentration of bupivacaine in the composition is from about 18 mg/ml to about 22 mg/mL, or about 20 mg/mL.

In some embodiments of the methods disclosed herein, the effective amount of bupivacaine administered is about 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 220 mg, 240 mg, 260 mg, 280 mg, or 300 mg, or a range defined by any two of the preceding values. In further embodiments, the effective amount of from about 10 mg to about 200 mg, from about 20 mg to about 150 mg, from 25 mg to about 100 mg, or from 30 mg to about 75 mg. In one embodiment, the amount of bupivacaine administered is about 66.5 mg. In another embodiment, the amount of bupivacaine administered is about 133 mg.

In some embodiments of the methods disclosed herein, the total volume of the bupivacaine MVL composition administered is about 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL or 20 mL, or a range defined by any two of the preceding values. In further embodiments, the total volume of the bupivacaine MVL composition administered is from about 1 mL to about 20 mL, from about 2 mL to about 15 mL, from about 3 mL to about 10 mL or from about 4 mL to about 8 mL, or about 5 mL. The total volume refers to the volume administered in a single dose in one area, for example, either to the left stellate ganglion or the right stellate ganglion. For bilateral SGB, if the volume administered to each side is about 5 mL, then the patient receives a total volume of 10 mL. In further embodiments, the total volume of the composition administered is from about 5 mL to about 15 mL for unilateral stellate ganglion block. In another embodiment, the total volume of the bupivacaine MVL composition administered is about 10 mL for unilateral stellate ganglion block. In further embodiments, the total volume of the composition administered is from about 10 mL to about 20 mL for bilateral stellate ganglion block. In one embodiment, the total volume of the composition administered is about 20 mL for bilateral stellate ganglion block.

In any embodiments of the method described herein, the composition of bupivacaine MVLs may extend the effect or duration of the SGB by at least 10%, 15%, 20%, 25%, 30%, 35%, 40%, 45%, 50%, 60%, 70%, 80%, 90%, or 100%, when compared to the administration of the same dose amount of bupivacaine in an immediate release composition.

### Methods of Administration

Some embodiments of the present application are related to methods of administering a composition comprising an effective amount of bupivacaine MVLs, as described herein, to one or more nerves of stellate ganglion of a patient, or an autonomic tissue area peripheral to the stellate ganglion. In some embodiments, a treatment apparatus comprises a needle and a compartment for the bupivacaine MVLs.

In some embodiments of the methods described herein, the administration includes navigating a treatment apparatus to stellate ganglion of the patient, or an autonomic tissue area peripheral to the stellate ganglion; injecting bupivacaine MVLs to one or more nerves of the stellate ganglion, or to one or more nerves of the autonomic tissue area peripheral to the stellate ganglion, or both; and removing the treatment apparatus from the patient.

In some embodiments of the methods described herein, navigating the treatment apparatus to the stellate ganglion includes inserting the needle of the treatment apparatus percutaneously to one or more nerves of the stellate ganglion area, or to one or more nerves of the autonomic tissue area peripheral to the stellate ganglion, or both. For example, the treatment apparatus may be inserted through an anterior wall of the trachea to tissue comprising a deep cardiac plexus. In some embodiments, navigating the treatment apparatus to the stellate ganglion includes guiding the needle using an imaging guide. In further embodiments, the imaging guide includes fluoroscopic or ultrasound imaging guide. The patient may be under general anesthesia or be intubated. In some embodiments, the administration is to the left stellate ganglion. In some embodiments, the administration is to the right stellate ganglion. In some embodiments, the administration is to both the left and the right stellate ganglion.

In some embodiments, the bupivacaine MVLs may be administered by bolus injection, continuous infusion, or a combination thereof. Administration of the instant bupivacaine

MVL composition may also be accomplished using standard methods and devices, e.g., injector systems, needle and syringe, a subcutaneous injection port delivery system, and the like. The administration of the bupivacaine MVLs composition may be used in conjunction with Pacira's handheld cryoanalgesia device iovera®.

### EXAMPLES

The following examples, including experiments and results achieved, are provided for illustrative purposes only and are not to be construed as limiting the present application.

### Example 1: Stellate Ganglion Blockade Procedure for Raynaud's Syndrome

In this example, a patient suffering from severe or refractory Raynaud's syndrome who hasn't responded well to other treatments like calcium channel blockers is selected. Bupivacaine encapsulated multivesicular liposomes (e.g., Exparel®) are injected anterior to the patient's longus colli muscle. Ultrasound guidance is used to introduce a 2.5 cm 25-gauge needle at the patient's C6 level for continuous infusion of bupivacaine MVLs of the present disclosure. The mean infusion rate is about 5 mL per hour. The needle is directed toward the longus colli muscle overlying Chassaignac's tubercle, following an oblique path medial to the common carotid artery and jugular vein.

### Example 2: Stellate Ganglion Blockade Procedure for Raynaud's Syndrome

In this example, a patient suffering from or at risk of suffering from Raynaud's syndrome is selected. Bupivacaine MVLs of the present disclosure are injected at C7. The point of injection is determined using the 2-finger method, located over the transverse notch at C7 and along the medial edge of the sternocleidomastoideole. Intradermal local anesthesia (e.g., 0.5 mL) is administered at the injection site first. The sternocleidomastoideole is moved inferiorly and laterally with the first and second fingers of one hand, such that the carotid sheath is laterally pulled. A 20G needle tip injector containing the bupivacaine encapsulated multivesicular liposomes such as Exparel® (e.g., 2 to 10 mL or 5 to 6 mL) is used to enter the skin vertically, and the needle is slowly advanced until the transverse notch of the C7 vertebra is felt. The block is reapplied once a day for 5 days on the same side.

### Example 3: Stellate Ganglion Blockade Procedure for Post-Mastectomy Pain Syndrome (PMPS)

In this example, patients suffering from or at risk of suffering from post-mastectomy pain syndrome are randomized into two groups: Group A receives ultrasound guided SGB one hour before surgery using bupivacaine MVLs (e.g., about 2-10 mL, or about 3 to 5 mL) of the present disclosure (such as Exparel®) and multimodal systemic analgesia, Group B (control) received multimodal systemic analgesia only. Ultrasound guidance is used to visualize Chassaignac's tubercle of C6 and differentiate it from C7. The ultrasound transducer is moved down to the C7 level, and a 22-gauge needle is inserted laterally with an in-plane approach, avoiding the jugular vein. A volume of NaCl solution (e.g., 1-2 mL of 0.9% NaCl w/v) is injected beneath or above the fascia covering the longus colli muscle to allow for visualization of the correct position, where bupivacaine MVLs are injected. Patients are followed up for six months. PMPS is assessed using the grading system for neuropathic pain (GSNP). Postoperative opioid consumption in the first 24 hours and numeric rating scale (NRS) are documented. Patient daily activity and functional capacity are evaluated using the Eastern Cooperative Oncology Group (ECOG) score. Following mastectomy with axillary dissection, preoperative US guided SGB is associated with less PMPS proportion, postoperative pain and opioid consumption and better patient daily activity and functional capacity.

### Example 4: Stellate Ganglion Blockade Procedure for Long COVID

In this example, a patient suffering from or at risk of suffering from long COVID is selected. On a first day, bupivacaine MVLs of the present disclosure are injected for a one-sided stellate ganglion block (e.g., right-sided or left-sided) with ultrasound guidance. 5mL to 10 mL of Exparel® may be injected. In addition, 1 ml (10 mg) dexamethasone may optionally be administered concurrently. The same SGB injection can be repeated after 3 days, 5 days, 7 days, 1 week, 2 weeks, 3 weeks or 4 weeks on the contralateral untreated side to further improve shortness of breath or GI issues.

While the present application has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter. process, process step or steps, to the objective, spirit and scope of the present invention. All such modifications are intended to be within the scope of the claims appended hereto.

1. A method of treating a patient who has suffered from or is at risk of suffering from a disorder associated with sympathetic nervous system, comprising:
administering a composition comprising an effective amount of bupivacaine multivesicular liposomes (MVLs) to one or more nerves of the stellate ganglion of the patient, or an autonomic tissue area peripheral to the stellate ganglion.

2. (canceled)

3. A method of treating or ameliorating an overactive or unbalanced sympathetic nervous system associated disorder in a patient in need thereof, comprising:
administering a composition comprising an effective amount of bupivacaine MVLs to one or more nerves of the stellate ganglion of the patient, or an autonomic tissue area peripheral to the stellate ganglion.

4. (canceled)

5. A method of reducing or interrupting sympathetic nervous system activity associated with a sympathetic nervous system disorder in a patient in need thereof, comprising:
administering a composition comprising an effective amount of bupivacaine MVLs to one or more nerves of the stellate ganglion of the patient, or an autonomic tissue area peripheral to the stellate ganglion.

6. (canceled)

7. The method of claim 1, wherein the sympathetic nervous system disorder is Raynaud's syndrome, post-mastectomy pain, reflex sympathetic dystrophy (RSD), phantom limb pain, hyperhidrosis, trigeminal neuralgia/facial pain, scleroderma, or long COVID.

8. (canceled)

9. (canceled)

10. (canceled)

11. (canceled)

12. (canceled)

13. (canceled)

14. (canceled)

15. (canceled)

16. The method of claim 1, wherein the method causes a temporary reduction in the ability of the sympathetic nervous system to release neurotransmitters.

17. The method of claim 1, wherein the one or more nerves of the stellate ganglion comprise one or more nerves that provide sympathetic nervous system activity associated with the sympathetic nervous system disorder.

18. The method of claim 1, wherein the concentration of bupivacaine in the composition is from about 1 mg/mL to about 30 mg/mL.

19. The method of claim 18, wherein the concentration of bupivacaine in the composition is about 13.3 mg/mL.

20. The method of claim 1, wherein the effective amount of bupivacaine administered is from about 10 mg to about 200 mg, from about 20 mg to about 100 mg, or from about 25 mg to about 75 mg.

21. The method of claim 1, wherein the total volume of the composition administered is from about 1 mL to about 20 mL.

22. The method of claim 21, wherein the total volume of the composition administered is from about 5 mL to about 10 mL for unilateral stellate ganglion block.

23. The method of claim 21, wherein the total volume of the composition administered is from about 10 mL to about 20 mL for bilateral stellate ganglion block.

24. (canceled)

25. The method of claim 1, wherein the bupivacaine is in the form of bupivacaine phosphate.

26. (canceled)

27. (canceled)

28. The method of claim 1, wherein the administering of the composition of bupivacaine MVLs comprises:
navigating a treatment apparatus to stellate ganglion of the patient, or an autonomic tissue area peripheral to the stellate ganglion, wherein the treatment apparatus comprises a needle and a compartment for the composition of bupivacaine MVLs;
injecting bupivacaine MVLs from the treatment apparatus to one or more nerves of the stellate ganglion, or the autonomic tissue area peripheral to the stellate ganglion; and
removing the treatment apparatus from the patient.

29. The method of claim 28, wherein navigating the treatment apparatus to the stellate ganglion comprises inserting the needle of the treatment apparatus percutaneously to one or more nerves of the stellate ganglion, or one or more nerves of the autonomic tissue area peripheral to the stellate ganglion.

30. (canceled)

31. (canceled)

32. The method of claim 28, wherein the injection comprises bolus injection, continuous infusion, or a combination thereof.

33. The method of claim 1, wherein the administering is to the left stellate ganglion.

34. The method of claim 1, wherein the administering is to both the left and the right stellate ganglion.

35. The method of claim 28, wherein the one or more nerves of the stellate ganglion or the autonomic tissue area peripheral to the stellate ganglion comprise at least one desired nerve selected from the group consisting of one or more desired nerves of the stellate ganglion, one or more desired nerves of the autonomic tissue area peripheral to the stellate ganglion, one or more desired nerves of a paravertebral chain, and combinations thereof.

36. (canceled)

37. (canceled)

38. The method of claim 35, wherein the one or more desired nerves of the paravertebral chain comprises one or more desired nerves of one or more of a C7 region of the paravertebral chain, a C8 region of the paravertebral chain, a T1 region of the paravertebral chain, or a T2 region of the paravertebral chain, or combinations thereof.

39. (canceled)
